(19) United States
(12) Patent Application Publication
(10) Pub. No.: US 2023/0346264 A1
(43) Pub. Date: Nov. 2, 2023

(54) INFORMATION PROCESSING DEVICE, INFORMATION PROCESSING SYSTEM, INFORMATION PROCESSING METHOD, AND INFORMATION PROCESSING PROGRAM

(71) Applicant: Sony Group Corporation, Tokyo (JP)

(72) Inventors: Takayoshi HIRAI, Tokyo (JP); Shin TAKANASHI, Tokyo (JP)

(73) Assignee: Sony Group Corporation, Tokyo (JP)

(21) Appl. No.: 18/246,202

(22) PCT Filed: Sep. 2, 2021
(86) PCT No.: PCT/JP2021/032250

§ 371 Date: Mar. 22, 2023

(30) Foreign Application Priority Data
Sep. 29, 2020 (JP) 2020-163443

(51) Int. Cl. A61B 5/113; G06F 3/01; A61B 5/00; G16H 10/60

(52) U.S. Cl. CPC A61B 5/113; G06F 3/011; A61B 5/0022; A61B 5/6844; A61B 5/746; G16H 10/60; G08C 17/02

(57) ABSTRACT

An information processing device (10) is an information processing device attached to a patient, and includes an acceleration sensor (13) (corresponding to an example of a “sensor”) that detects movement of the patient which movement is associated with respiratory movement, an acquisition unit (163a) that acquires sensor data of the acceleration sensor (13), an estimation unit (163b) that estimates a respiratory condition of the patient from the sensor data, and a determination unit (163c) that determines whether to transmit information related to the respiratory condition to an external device on the basis of the estimated respiratory condition.

### FIELD

The present disclosure relates to an information processing device, an information processing system, an information processing method, and an information processing program.

### BACKGROUND

Conventionally, in monitoring of a respiratory condition of a patient, a method of performing pulse wave analysis by pasting an electrode on the patient, and of measuring a blood oxygen level has been known. However, these methods have problems that it is necessary to accurately stretch and attach the electrode to the patient, and there is a time lag in a change between the respiratory condition and the blood oxygen level.

Thus, a method of monitoring a respiratory condition of a patient by measuring movement of a patient, such as chest movement or abdominal movement associated with respiratory movement with an acceleration sensor attached to the patient, for example, by being pasted has been proposed.

### CITATION LIST

### Patent Literature

Patent Literature 1: JP 2006-320732 A

### SUMMARY

### Technical Problem

However, the above-described conventional technology has room for further improvement in monitoring a respiratory condition of a patient while controlling power consumption and securing a real-time property.

For example, in a case where an acceleration sensor is attached to the patient, it is preferable that the sensor is a wireless type in consideration of wiring disconnection or the like due to a change in a posture of the patient. However, in a case of the wireless type, in order to secure the real-time property, wireless connection to an external device that performs monitoring is constantly necessary and the power consumption is increased.

In this regard, in the above-described conventional technology, sensor data is collected to a recording device attached to the patient together with the acceleration sensor, and is collectively transferred to an aggregation device on the basis of operation by a user. However, such a method cannot secure the real-time property.

Thus, the present disclosure proposes an information processing device, information processing system, information processing method, and information processing program capable of monitoring a respiratory condition of a patient while controlling power consumption and securing a real-time property.

### Solution to Problem

In order to solve the above problems, one aspect of an information processing device according to the present disclosure is an information processing device attached to a patient, and includes a sensor that detects movement of the patient which movement is associated with respiratory movement, an acquisition unit that acquires sensor data of the sensor, an estimation unit that estimates a respiratory condition of the patient from the sensor data, and a determination unit that determines whether to transmit information related to the respiratory condition to an external device on the basis of the estimated respiratory condition.

### BRIEF DESCRIPTION OF DRAWINGS

FIG. 1 is a view illustrating a configuration example of an information processing device according to an embodiment of the present disclosure.

FIG. 2 is a view for describing an outline of an information processing method according to the embodiment of the present disclosure.

FIG. 3 is a view illustrating a modification example of a method of attachment to a patient.

FIG. 4 is a view illustrating a configuration example of an information processing system according to the embodiment of the present disclosure.

FIG. 5 is a view illustrating an example of user information.

FIG. 6 is a block diagram illustrating the configuration example of the information processing device according to the embodiment of the present disclosure.

FIG. 7 is a block diagram illustrating a configuration example of an integration server according to the embodiment of the present disclosure.

FIG. 8 is a first view for describing learning processing.

FIG. 9 is a second view for describing the learning processing.

FIG. 10 is a view illustrating an example of output information to be output to a terminal device.

FIG. 11 is a flowchart illustrating a processing procedure of the information processing device according to the embodiment of the present disclosure.

FIG. 12 is a flowchart illustrating a processing procedure of an information processing device according to a first modification example.

FIG. 13 is a hardware configuration diagram illustrating an example of a computer that realizes functions of an information processing device.

### DESCRIPTION OF EMBODIMENTS

In the following, embodiments of the present disclosure will be described in detail on the basis of the drawings. Note that in each of the following embodiments, overlapped description is omitted by assignment of the same reference sign to the same parts.

Also, the present disclosure will be described in the following order of items.
1. Outline of an embodiment of the present disclosure
2. Configuration of an information processing system
2-1. Overall configuration
2-2. Configuration of an information processing device
2-3. Configuration of an integration server
3. Processing procedure of the information processing device
4. Modification example
4-1. First modification example
4-2. Second modification example
4-3. Third modification example
4-4. Fourth modification example
4-5. Fifth modification example
4-6. Sixth modification example
4-7. Other modification examples
5. Hardware configuration
6. Conclusion

### 1. Outline of an Embodiment of the Present Disclosure

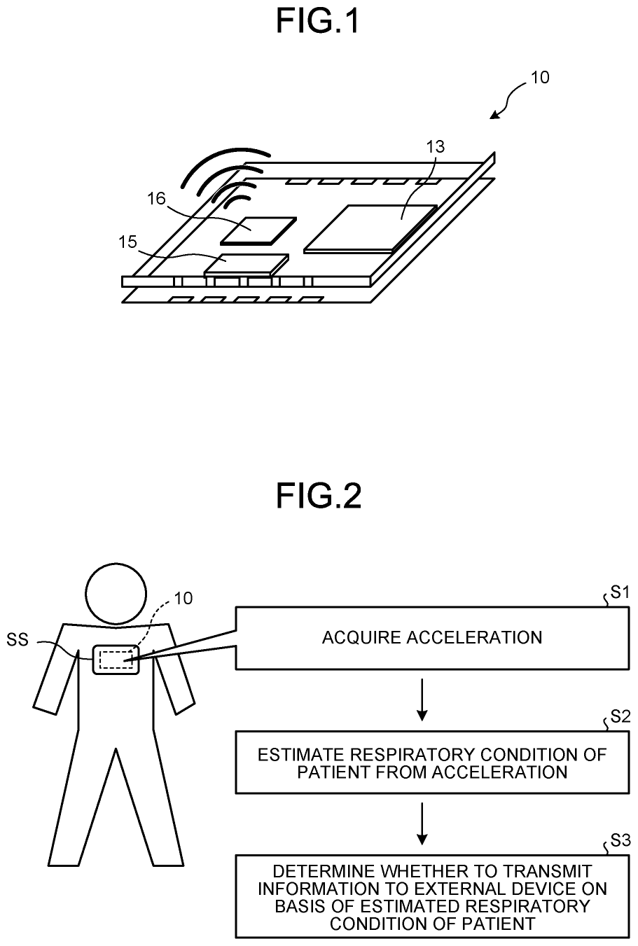

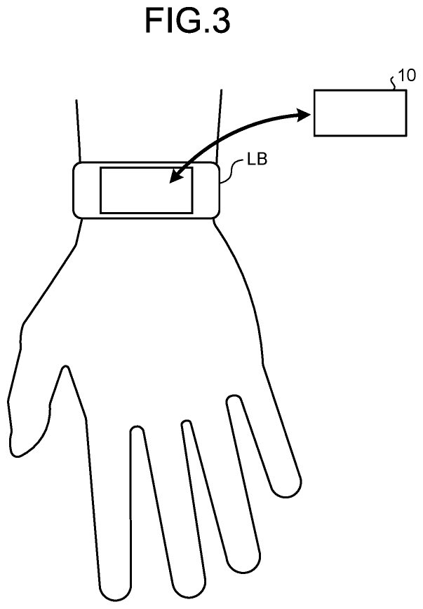

FIG. 1 is a view illustrating a configuration example of an information processing device 10 according to the embodiment of the present disclosure. Furthermore, FIG. 2 is a view for describing an outline of an information processing method according to the embodiment of the present disclosure. In addition, FIG. 3 is a view illustrating a modification example of a method of attachment to a patient.

The information processing method according to the embodiment of the present disclosure relates to monitoring of a respiratory condition of a patient with an information processing device 10 that includes a sensor to detect movement of the patient, the movement being associated with respiratory movement, and that is attached to the patient, that is, the information processing device 10 that can also be referred to as an edge sensor.

As illustrated in FIG. 1, the information processing device 10 includes an acceleration sensor 13, a battery 15, and a microcomputer 16. The acceleration sensor 13, the battery 15, and the microcomputer 16 are mounted on, for example, a printed circuit board or the like.

The acceleration sensor 13 is an example of a sensor to detect movement of the patient, which movement is associated with the respiratory movement, and measures acceleration by chest movement, abdominal movement, or the like associated with the respiratory movement. The battery 15 supplies power to the acceleration sensor 13 and the microcomputer 16. The microcomputer 16 includes a wireless communication chip (not illustrated) and is provided in a manner of being able to wirelessly communicate with an external device.

Incidentally, in recent years, it has been found that a patient who goes into cardiac arrest exhibits some abnormal symptoms or signs in advance. For example, 70% of cardiac arrest are said to exhibit evidence of exacerbation of respiratory symptoms within eight hours prior to the cardiac arrest. Thus, monitoring of the respiratory condition is demanded to be performed with an edge sensor that can be applied not only to a severe patient but also to various patients and that is easier to be operated.

That is, the edge sensor is preferably a wireless-type as with the information processing device 10 in consideration of wiring disconnection due to a change in a posture of the patient, hindrance of movement of the patient, and the like. However, in a case of the wireless type, in order to secure a real-time property of the monitoring, wireless connection to an external device is constantly necessary and power consumption is increased.

In this regard, in the conventional technology, a method of collecting sensor data into a recording device attached to the patient together with the acceleration sensor 13, and collectively transferring the sensor data to an aggregation device on the basis of operation by a user is employed. However, such a method cannot secure the real-time property of the monitoring.

Thus, in the information processing method according to the embodiment, the information processing device 10 acquires sensor data of the acceleration sensor 13, estimates a respiratory condition of the patient from the sensor data, and determines whether to transmit information related to the respiratory condition to an external device on the basis of the estimated respiratory condition.

Specifically, as the wireless communication chip described above, first, the information processing device 10 includes a wireless communication chip compliant with, for example, a low power, wide area (LPWA) standard. The LPWA system realizes wireless communication characterized by low power consumption, a low bit rate, and wide area coverage.

Note that, in the present embodiment, the information processing device 10 performs wireless communication with the external device by the LPWA system, and a wireless communication chip included in the microcomputer 16 is hereinafter referred to as an “LPWA communication unit 161”.

Then, as illustrated in FIG. 2, the information processing device 10 including the LPWA communication unit 161 is attached to the patient by being pasted on the chest, abdomen, or the like of the patient by utilization of, for example, an adhesive sheet SS.

Note that as illustrated in FIG. 3, the information processing device 10 may be detachably provided in a wristband LB worn on the patient, and may be attached to a wrist of the patient. As described above, the information processing device 10 preferably has a structure in which a main body portion is separable. As a result, sterilization and disinfection by an autoclave can be easily performed. In addition, it becomes possible to perform flexible operation such as pasting on the chest or abdomen of the patient, mounting on the wristband LB, and attaching to a chest pocket of the patient.

Then, by utilization of the information processing device 10 attached to the patient in such a manner, in the information processing method according to the embodiment, the information processing device 10 acquires acceleration as illustrated in FIG. 2 (Step S1). Then, the information processing device 10 estimates a respiratory condition of the patient from the acquired acceleration (Step S2).

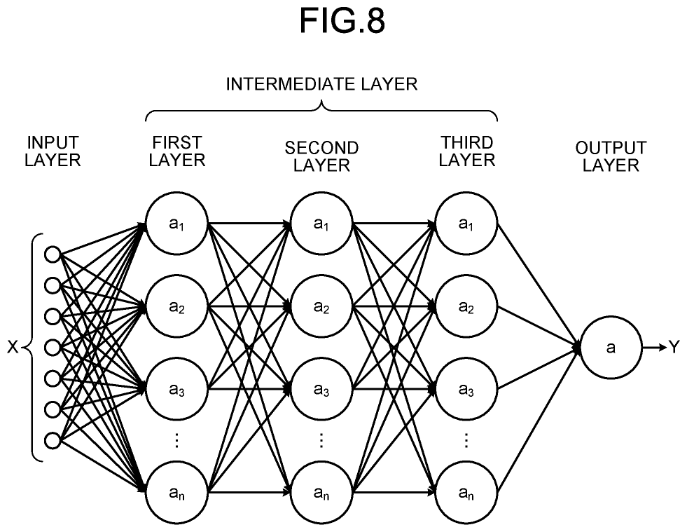

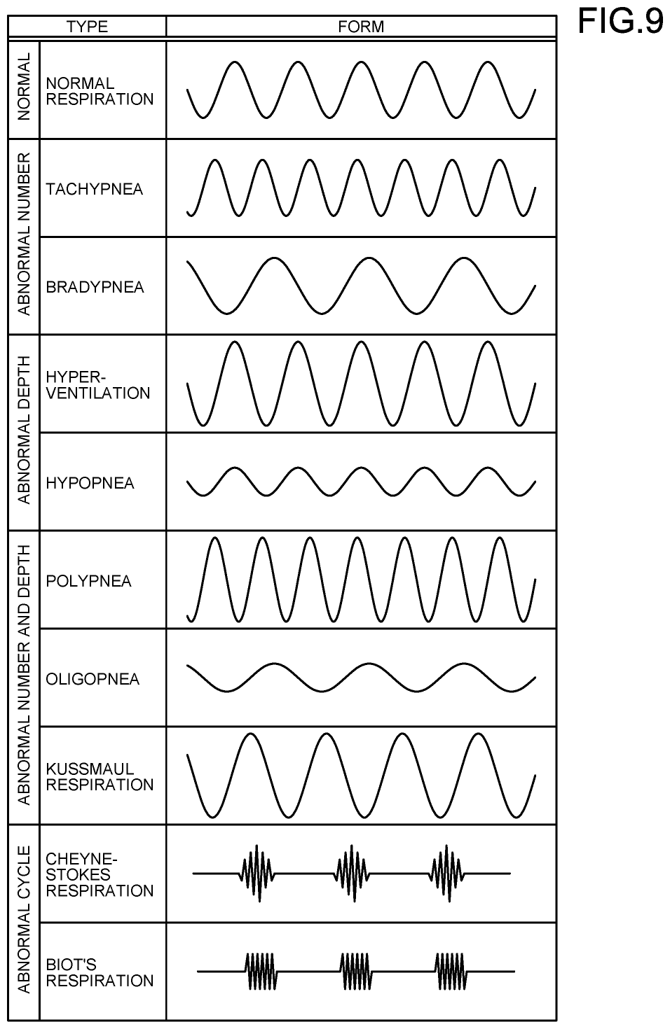

Note that in Step S2, the information processing device 10 estimates the respiratory condition by using an estimation model generated by machine learning, for example. Such a point will be described later with reference to FIG. 8 and FIG. 9.

Then, the information processing device 10 determines whether to transmit information related to the respiratory condition to the external device on the basis of the estimated respiratory condition of the patient (Step S3). For example, in a case where it is estimated that there is abnormality in the respiratory condition of the patient, the information processing device 10 determines to transmit the information to the external device. Then, the information processing device 10 causes the LPWA communication unit 161 and the external device to be wirelessly connected, and causes the LPWA communication unit 161 to transmit the information related to the respiratory condition, such as notification indicating presence of abnormality and the acquired acceleration.

Furthermore, for example, in a case where it is estimated that there is no abnormality in the respiratory condition of the patient, the information processing device 10 determines not to transmit the information until a predetermined transmission cycle comes, and does not cause the LPWA communication unit 161 and the external device to be connected wirelessly while the transmission is not performed. Note that the information processing device 10 continuously repeats the acquisition of the acceleration in Step S1 and the estimation of the respiratory condition in Step S2 even while the information is not transmitted.

Then, when there is no abnormality in the respiratory condition and the predetermined transmission cycle comes, the information processing device 10 causes the LPWA communication unit 161 and the external device to be wirelessly connected, and causes the LPWA communication unit 161 to transmit only a notification indicating that there is no abnormality, for example.

In such a manner, in the information processing method according to the embodiment, the information processing device 10 acquires the sensor data of the acceleration sensor 13, estimates the respiratory condition of the patient from the sensor data, and determines whether to transmit the information related to the respiratory condition to the external device on the basis of the estimated respiratory condition.

In other words, in the information processing method according to the embodiment, a side of the information processing device 10 that is the wireless-type edge sensor estimates the respiratory condition on the basis of the acceleration acquired in real time, and appropriately transmits necessary data when necessary on the basis of a result of the estimation. In addition, the LPWA communication unit 161 that is compliant with the LPWA standard is used to transmit such data.

Thus, according to the information processing method of the embodiment, it is possible to monitor the respiratory condition of the patient while controlling the power consumption and securing the real-time property.

Hereinafter, a configuration example of an information processing system 1 to which the information processing method according to the above-described embodiment is applied will be described more specifically.

### 2. Configuration of an Information Processing System

### 2-1. Overall Configuration

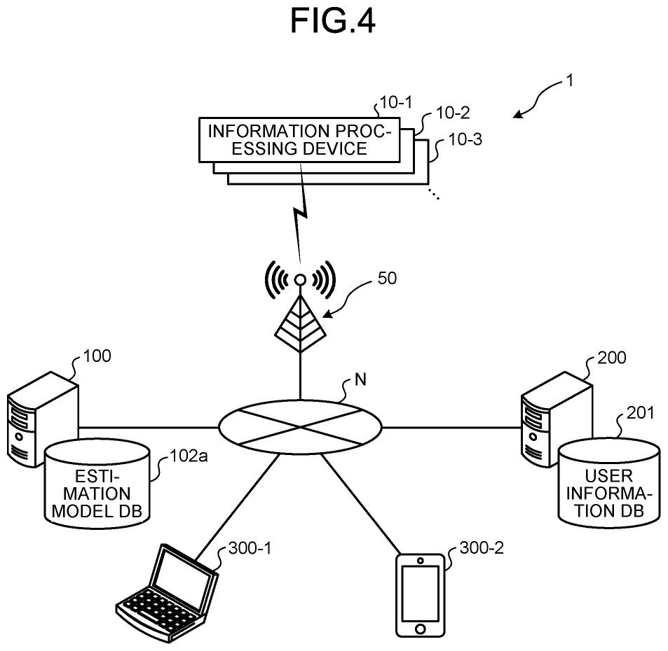

First, an overall configuration of the information processing system 1 will be described. FIG. 4 is a view illustrating a configuration example of the information processing system 1 according to the embodiment of the present disclosure. As illustrated in FIG. 4, the information processing system 1 includes one or more information processing devices 10(10-1, 10-2, 10-3 . . .), a base station device 50, an integration server 100, an information server 200, and one or more terminal devices 300 (300-1, 300-2 . . . ).

The one or more information processing devices 10 and the base station device 50 are wirelessly connected at the time of communication, and transmit and receive information via LPWA communication. In addition, the base station device 50, the integration server 100, the information server 200, and the one or more terminal devices 300 are mutually connected by a network N, and transmit and receive information to and from each other via the network N. The network N is a public line, a dedicated line, a wireless line, a wired line, or the like, or the Internet in which a plurality of these lines are connected to each other, or the like. Each of the information processing devices 10 transmits and receives information to and from the integration server 100, the information server 200, and the one or more terminal devices 300 via the base station device 50.

Since the outline of the information processing device 10 has been described, a description thereof will be simplified here. The information processing device 10 functions as the edge sensor including the acceleration sensor 13 as described above. As described above, the acceleration sensor 13 is an example of a sensor to detect movement of the patient, which movement is associated with the respiratory movement, and measures the acceleration caused by the chest movement, the abdominal movement, movement of an arm, or the like associated with the respiratory movement, in other words, the acceleration caused by an increase or decrease in a bulge of the lung.

Note that although the acceleration sensor 13 is used in the present embodiment, an angular velocity sensor or a magnetic sensor may be used as a sensor that detects movement of the patient which movement is associated with the respiratory movement. In addition, a blood oxygen level sensor that measures a blood oxygen level may be used in a sense of measuring movement in blood of the patient which movement is associated with the respiratory movement.

The base station device 50 is a device serving as a local base station of the information processing device 10, and has a function as a relay base station and a function of protocol conversion. The base station device 50 is provided within a reaching distance from the information processing device 10 via the LPWA communication, and is provided in a hospital room, for example.

The integration server 100 is a server device that acquires and processes sensor data and sensor information (such as sensor ID) transmitted from the information processing device 10. The integration server 100 acquires user information that is information related to the patient from the information server 200, generates output information on the basis of the sensor data, the sensor information, and the user information, and outputs the output information to the one or more terminal devices 300.

In addition, the integration server 100 includes an estimation model database (DB) 102a. The estimation model DB 102a is a database of an estimation model used by the information processing device 10 to estimate the respiratory condition of the patient. For example, the integration server 100 generates the estimation model to estimate the respiratory condition of the patient from the acceleration by machine learning and performs management thereof by the estimation model DB 102a. In addition, the integration server 100 distributes the generated estimation model to each of the information processing devices 10.

The information server 200 is a server device that manages the above-described user information registered in advance. The information server 200 includes a user information DB 201. The user information DB 201 stores the user information. The user information is information in which information for specifying the patient (such as user ID) and information for specifying the information processing device 10 (such as sensor ID) are associated with each other.

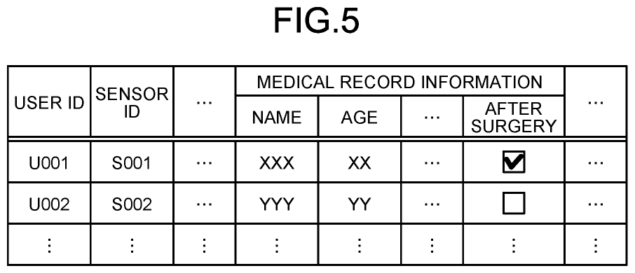

FIG. 5 is a view illustrating an example of the user information. Specifically, as illustrated in FIG. 5, the user information is information in which a “user ID” and a “sensor ID” are associated with each other. In the user information, “medical record information” of each patient is further associated with the “user ID” and the “sensor ID”.

The “medical record information” includes items such as a “name” and an “age” of each patient, and “after surgery” in which a flag value indicating whether the user is immediately after surgery is stored. An example of processing using such “medical record information” will be described later.

The description returns to FIG. 4. Each of the terminal devices 300 is information equipment used by each of medical workers such as a doctor, a nurse, and a care worker. The terminal device 300 is a desktop personal computer (PC), a notebook PC, a mobile phone including a smartphone, a tablet terminal, a personal digital assistant (PDA), or the like. Furthermore, the terminal device 300 may be, for example, a wearable terminal or the like.

### 2-2. Configuration of an Information Processing Device

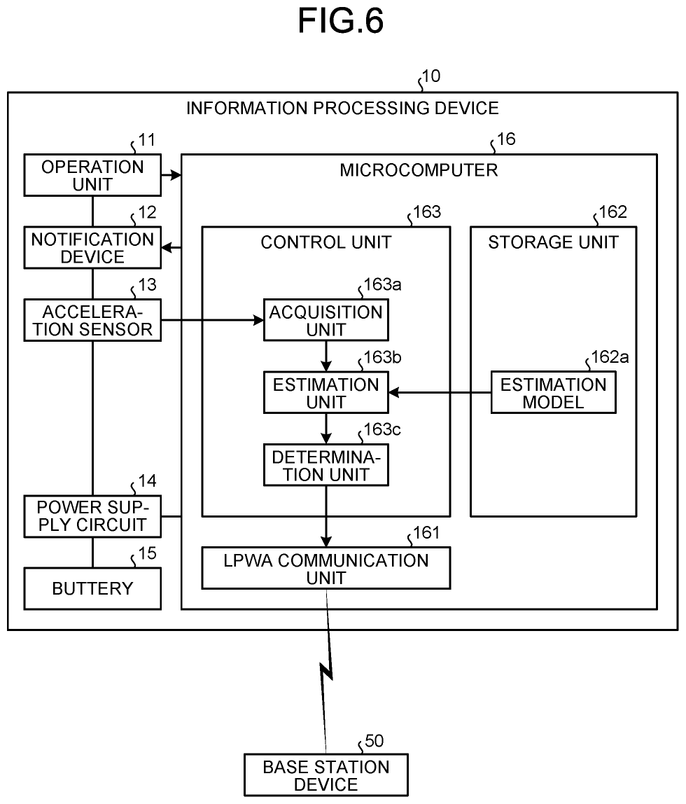

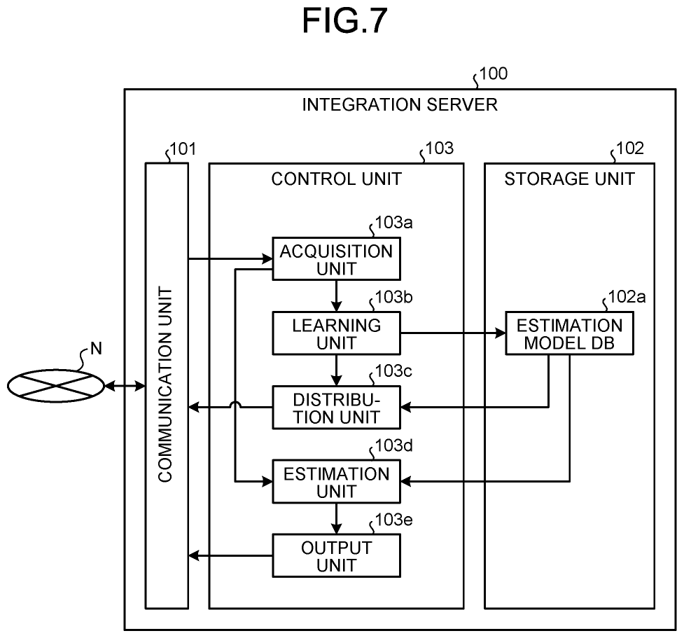

Next, the configuration example of each of the information processing devices 10 will be described. FIG. 6 is a block diagram illustrating the configuration example of the information processing device 10 according to the embodiment of the present disclosure. Note that only components necessary for describing features of the present embodiment are illustrated in FIG. 6 and FIG. 7 (described later), and a description of general components is omitted.

In other words, each of the components illustrated in FIG. 6 and FIG. 7 is functionally conceptual, and is not necessarily configured physically in an illustrated manner. For example, a specific form of distribution/integration of each block is not limited to what is illustrated in the drawings, and a whole or part thereof can be functionally or physically distributed/integrated in an arbitrary unit according to various loads and usage conditions.

Furthermore, in the description using FIG. 6 and FIG. 7, a description of the already-described components may be simplified or omitted.

The information processing device 10 is configured as a so-called event-detection-type edge sensor. As illustrated in FIG. 6, the information processing device 10 includes an operation unit 11, a notification device 12, an acceleration sensor 13, a power supply circuit 14, a battery 15, and a microcomputer 16.

The operation unit 11 is, for example, a switch to turn ON/OFF a power supply, to perform transmission, and to notify a state. The notification device 12 is a device to notify an operation state of the information processing device 10, and is realized by, for example, a light emitting diode (LED) or the like.

The acceleration sensor 13 measures the acceleration generated by the respiratory condition of the patient, as described above. Note that the acceleration sensor 13 can preferably measure acceleration in three-axis directions, and is preferably a three-axis micro electro mechanical systems (MEMS) acceleration sensor.

The power supply circuit 14 controls the battery 15. The power supply circuit 14 supplies power from the battery 15 to the operation unit 11, the notification device 12, the acceleration sensor 13, and the microcomputer 16. The battery 15 is a power supply controlled by the power supply circuit 14, and is a rechargeable lithium battery, for example.

Note that the power supply circuit 14 is preferably a circuit capable of supplying power wirelessly. As a result, it is possible to reduce labor of a medical worker at the time of charging. In addition, since no power supply outlet is necessary, sterilization and disinfection by, for example, an autoclave can be easily performed.

The microcomputer 16 is a main processing unit of the information processing device 10. The microcomputer 16 includes the LPWA communication unit 161, a storage unit 162, and a control unit 163.

Although the LPWA communication unit 161 has already been described, the description is supplemented. The LPWA communication unit 161 is a wireless communication chip suitable for use in a case where communication is frequently performed although power consumption is extremely small and a data size is small.

As a system of the LPWA communication, any system may be used although a plurality of systems is known. Preferably, a system with a long reaching distance is preferred (about several tens of kilometers to 100 km). Note that a system other than the LPWA communication, or a system of a different wavelength may be used as the communication system. Furthermore, in a case where bidirectional communication is performed, a wavelength different from the LPWA may be used as a reception wave.

The storage unit 162 is realized by, for example, a semiconductor memory element such as a random access memory (RAM), a read only memory (ROM), or a flash memory. In the example illustrated in FIG. 6, the storage unit 162 stores an estimation model 162a.

As described above, the estimation model 162a is a machine learning model that is generated by machine learning and that estimates the respiratory condition of the patient on the basis of the acceleration. The estimation model 162a is generated by the integration server 100 and stored in advance in the information processing device 10. In addition, the estimation model 162a is distributed from the integration server 100, for example, in a case of being updated by relearning or the like.

The control unit 163 is a controller, and is realized, for example, when various programs stored in the storage unit 162 are executed by a central processing unit (CPU), a micro processing unit (MPU), or the like with a RAM as a work area. Also, the control unit 163 can be realized by, for example, an integrated circuit such as an application specific integrated circuit (ASIC) or a field programmable gate array (FPGA).

The control unit 163 includes an acquisition unit 163a, an estimation unit 163b, and a determination unit 163c, and realizes or executes a function and action of information processing described in the following.

The acquisition unit 163a measures the acceleration measured by the acceleration sensor 13. Note that the acquisition unit 163a acquires the acceleration for a predetermined time (such as 30 seconds).

The estimation unit 163b estimates the respiratory condition of the patient on the basis of the acceleration acquired by the acquisition unit 163a. Specifically, the estimation unit 163b inputs the acceleration acquired by the acquisition unit 163a to the estimation model 162a, and acquires an output value output from the estimation model 162a on the basis of the input.

For example, the estimation model 162a outputs, as the output value, a type value indicating whether the respiratory condition of the patient is abnormal or a type value indicating a type of abnormality in a case where there is the abnormality. The estimation unit 163b outputs the output value acquired from the estimation model 162a to the determination unit 163c.

On the basis of the output value output from the estimation unit 163b, the determination unit 163c determines whether to transmit the information related to the respiratory condition of the patient to the external device, that is, the integration server 100.

Specifically, in a case where the respiratory condition is normal, the determination unit 163c causes the LPWA communication unit 161 to transmit notification indicating normality to the integration server 100 via the base station device 50 for a predetermined number of times (such as once) in a predetermined period (such as five minutes). Note that the sensor data may be transmitted together at this time.

On the other hand, in a case where the respiratory condition is abnormal, the determination unit 163c causes the LPWA communication unit 161 to transmit the notification indicating abnormality (such as type value of abnormality described above) and the sensor data to the integration server 100 without waiting for the predetermined period.

As a result, when an event that the respiratory condition is abnormal is not generated (that is, when the respiratory condition is normal), the number of times of transmitting the information can be reduced. Thus, power used for the wireless transmission can be reduced.

When the normal respiratory condition continues for a predetermined period (such as 30 minutes), a cycle of estimating the respiratory condition may be extended from once in every five minutes to once in every ten minutes, for example. This makes it possible to reduce power used for the estimation processing of the respiratory condition. Note that it is preferable to keep acquiring the acceleration itself.

In addition, in a case where the event that the respiratory condition is abnormal is generated, the determination unit 163c transmits not only the notification indicating that there is the abnormality but also the sensor data used to estimate the respiratory condition to integration server 100 as described above. As a result, it is possible to cause a side of the integration server 100 to verify whether the respiratory condition estimated by the side of the information processing device 10 is correct, or to determine the respiratory condition by using a more accurate machine learning model.

### 2-3. Configuration of an Integration Server

Next, a configuration example of the integration server 100 will be described. FIG. 7 is a block diagram illustrating the configuration example of the integration server 100 according to the embodiment of the present disclosure.

As illustrated in FIG. 7, the integration server 100 includes a communication unit 101, a storage unit 102, and a control unit 103.

The communication unit 101 is realized, for example, by a network interface card (NIC) or the like. The communication unit 101 is connected to the network N in a wireless or wired manner, and transmits and receives information to and from the base station device 50 (that is, information processing device 10), the information server 200, and the terminal device 300 that are also connected to the network N.

The storage unit 102 is realized, for example, by a semiconductor memory element such as a RAM, ROM, or flash memory, or a storage device such as a hard disk or optical disk. In the example illustrated in FIG. 7, the storage unit 102 stores the estimation model DB 102a described above.

The control unit 103 is a controller, and is realized, for example, when various programs stored in the storage unit 162 are executed by the CPU, MPU, or the like with the RAM as a work area. Furthermore, the control unit 103 can be realized by, for example, an integrated circuit such as an ASIC or an FPGA.

The control unit 103 includes an acquisition unit 103a, a learning unit 103b, a distribution unit 103c, an estimation unit 103d, and an output unit 103e, and realizes or executes a function and action of information processing described in the following.

First, processing operation at the time of learning in the integration server 100 will be described. Via the communication unit 101 or an input device (not illustrated), the acquisition unit 103a acquires a learning data set of acceleration which data set is to be teacher data in machine learning. In addition, the acquisition unit 103a outputs the acquired learning data set to the learning unit 103b.

The learning unit 103b executes machine learning using the learning data set input from the acquisition unit 103a, generates an estimation model for estimating the respiratory condition of the patient from the acceleration, and stores the estimation model into the estimation model DB 102a.

Here, learning processing executed by the learning unit 103b will be specifically described with reference to FIG. 8 and FIG. 9. FIG. 8 is a first view for describing the learning processing. Furthermore, FIG. 9 is a second view for describing the learning processing.

As illustrated in FIG. 8, the learning unit 103b executes the learning processing using a machine learning algorithm using a multilayer neural network, for example. As illustrated in FIG. 8, the multilayer neural network includes an input layer, intermediate layers, and an output layer. Note that although an example in which the intermediate layers are three layers of a first layer to a third layer is illustrated in FIG. 8, the number of layers is not limited.

The multilayer neural network performs an output Y with respect to an input X. When it is assumed that the acceleration in the three-axis directions acquired as the learning data set is aₓ, a_{y}, and a_{z} and resultant acceleration data is a, the learning unit 103b calculates the resultant acceleration data a by the following expression (1).

$$a = \sqrt{{a}_{x}^{2} + {a}_{y}^{2} + {a}_{z}^{2}} \qquad ( 1 )$$

In addition, the learning unit 103b generates seven kinds of feature amounts such as an average, variance, skewness, a kurtosis, signal power, a zero crossing number, and a maximum peak frequency by using the resultant acceleration data a. When a sampled sensor signal is represented by xᵢ and a signal sequence including N signals is described as X={xᵢ|i=1, 2, . . . , N}, each feature amount is calculated as follows.

For example, the average is one of basic statistics. The average of the signal sequence X is calculated by the following expression (2).

$$\overline{x} = \frac{1}{N} {\sum }_{i = 1}^{N} {x}_{i} \qquad ( 2 )$$

In addition, the variance is one of the basic statistics as well as the average, and is a measure indicating how far the signal sequence is from an average value. Variance σ² of the signal sequence X is calculated by the following expression (3).

$${σ}^{2} = \frac{1}{N} {\sum }_{i = 1}^{N} {( {x}_{i} - \overline{x} )}^{2} \qquad ( 3 )$$

Furthermore, the skewness is a statistic based on a third-order moment representing asymmetry of a distribution of the signal sequence, and is calculated by the following expression (4).

$${β}_{1} = \frac{1}{N} {\sum }_{i = 1}^{N} {( \frac{{x}_{i} - \overline{x}}{σ} )}^{3} \qquad ( 4 )$$

Here, skewness β₁ indicates a normal distribution when β₁=0, a right-skewed distribution when β₁>0, and a left-skewed distribution when β₁<0.

Furthermore, the kurtosis is a statistic based on a fourth-order moment representing sharpness of the distribution of the signal sequence, and is calculated by the following expression (5).

$$\overline{{x}^{2}} = \frac{1}{N} {\sum }_{i = 1}^{N} {x}_{i}^{2} \qquad ( 5 )$$

Here, kurtosis β₂ indicates a normal distribution when β₂=3, a long-tailed distribution with a sharp peak when β₂>3, and a short-tailed distribution with a rounded peak when β₂<3.

In addition, regarding the signal power, generally, magnitude of a time-axis signal is defined by a root mean square value. The signal power is calculated by the following expression (6).

$${β}_{2} = \frac{1}{N} {\sum }_{i = 1}^{N} {( \frac{{x}_{i} - \overline{x}}{σ} )}^{4} \qquad ( 6 )$$

The zero crossing number is the number of times a signal crosses a zero level in a certain time. In addition, the maximum peak frequency is a frequency at which a peak is the maximum in a power spectrum acquired by Fourier transform of the acceleration data.

The learning unit 103b calculates these plurality of feature amounts with respect to the acceleration in the predetermined period. For example, the learning unit 103b makes one set of learning data sets by associating a plurality of feature amounts with respect to the acceleration for five minutes with a condition of the patient for the five minutes. With this one set as X, the learning unit 103b inputs X={n₁, n₂, . . . } (n is a feature amount, for example, n₁ being an average, n₂ being variance . . . , and the like.) to the multilayer neural network.

Then, the learning unit 103b acquires the parameter aₓ of the intermediate layers by inputting all sets of the learning data sets to the multilayer neural network. As a result, it is possible to generate an estimation model that is a classifier that outputs a respiratory condition Y of the patient with respect to the input X.

Note that the estimation model 162a mounted on the information processing device 10 may be a machine learning model that uses the parameters acquired in the learning process as they are, or may be a calculation model that is a lighter machine learning model based on the parameters acquired in the learning process.

This is a method called pruning. The number of intermediate layers is increased and calculation is performed in the machine learning in the integration server 100, and a calculation amount can be reduced by deleting of an intermediate layer having a low influence on accuracy in the information processing device 10.

Note that the respiratory condition may be classified into two that are being normal and being abnormal, or may be classified into at least one of normal respiration, tachypnea, bradypnea, hyperventilation, hypopnea, polypnea, oligopnea, Kussmaul respiration, Cheyne-Stokes respiration, or Biot's respiration illustrated in FIG. 9.

In such a case, a side of a medical worker may be allowed to set which respiratory condition is abnormal, or a condition other than the normal respiration may be set to abnormal.

In addition, relearning and additional learning may be appropriately performed not only on the basis of the learning data set acquired in advance but also on the basis of the acceleration actually acquired from the patient and the respiratory condition of the patient actually acquired by the medical worker through monitoring or by another sensor such as a blood oxygen level sensor.

The description returns to FIG. 7. The distribution unit 103c distributes the estimation model generated by the learning unit 103b to each of the information processing devices 10 via the communication unit 101. Note that the distribution unit 103c may distribute the estimation model to each base station device 50, and the estimation model 162a may be downloaded from the base station device 50 to the information processing device 10 when the information processing device 10 and the base station device 50 are wirelessly connected.

Next, processing operation at the time of estimation of the respiratory condition in the integration server 100 will be described. In such a case, the acquisition unit 103a acquires information, which is related to the respiratory condition of the patient and which is transmitted from the information processing device 10, as needed. In addition, the acquisition unit 103a outputs the acquired information to the estimation unit 103d.

On the basis of the information input from the acquisition unit 103a, the estimation unit 103d estimates the respiratory condition of the patient by using any of the estimation models stored in the estimation model DB 102a. At this time, the estimation unit 103d estimates the respiratory condition by using, for example, a more accurate estimation model in such a manner that it is possible to determine whether the respiratory condition estimated on the side of the information processing device 10 is correct. Furthermore, the estimation unit 103d outputs a result of the estimation to the output unit 103e.

The output unit 103e generates output information to be transmitted to the terminal device 300 on the basis of the result of the estimation by the estimation unit 103d, and transmits the output information via the communication unit 101.

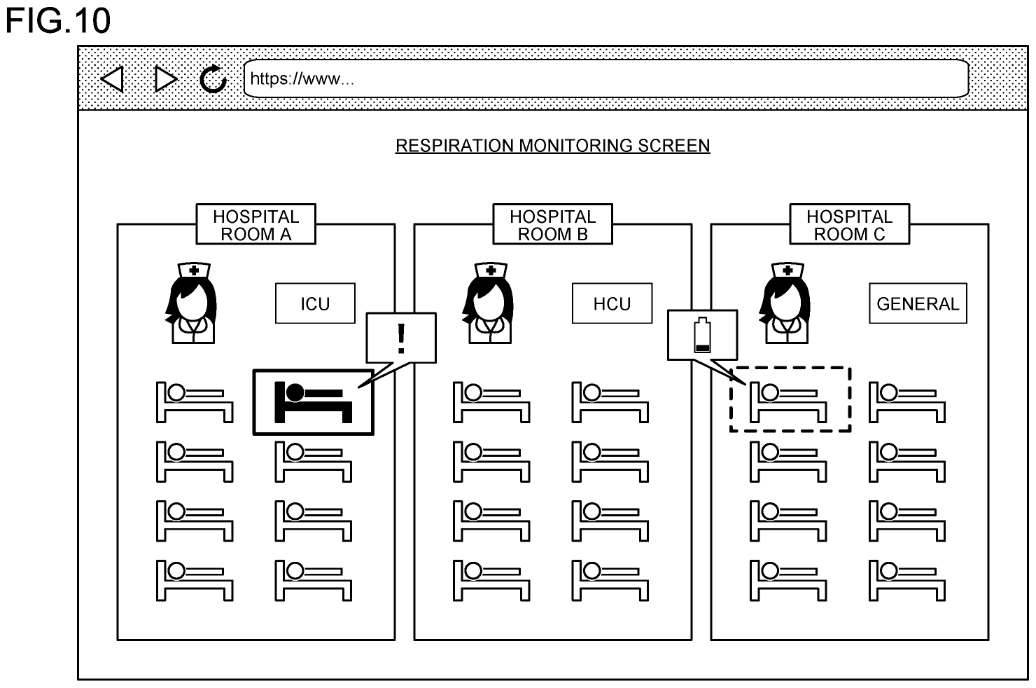

FIG. 10 is a view illustrating an example of the output information to be output to the terminal device 300. As the output information for the terminal device 300, the output unit 103e generates and outputs a user interface (UI) screen in a manner illustrated in FIG. 10, for example.

Via such a UI screen referred to, for example, as a “respiration monitoring screen”, the medical worker can integrally manage respiratory conditions of patients with the terminal device 300. In the “respiration monitoring screen”, for example, each patient is represented by each icon schematically representing a state of lying on a bed. For a patient with a normal respiratory condition, the icon is displayed in green, for example.

Furthermore, for a patient with an abnormal respiratory condition, the icon is displayed in red, for example. In addition, as illustrated in FIG. 10, the red icon is surrounded by, for example, a rectangular frame line and is further emphasized by an “!” mark.

When the medical worker clicks such the “!” mark, detailed information of the abnormal respiratory condition can be displayed, and it can be determined whether to perform a more detailed analysis on the side of the integration server 100.

Furthermore, not only the respiratory condition but also a mark indicating that charging is necessary, a broken rectangular frame line, and the like may be displayed together with the icon of the patient to whom the information processing device 10 requiring charging of the battery 15 is attached, as illustrated in FIG. 10. Note that charge information indicating whether charging is necessary is included in the sensor data transmitted from the information processing devices 10, and the side of the integration server 100 can grasp charge states of the batteries 15 of all the information processing devices 10.

### 3. Processing Procedure of the Information Processing Device

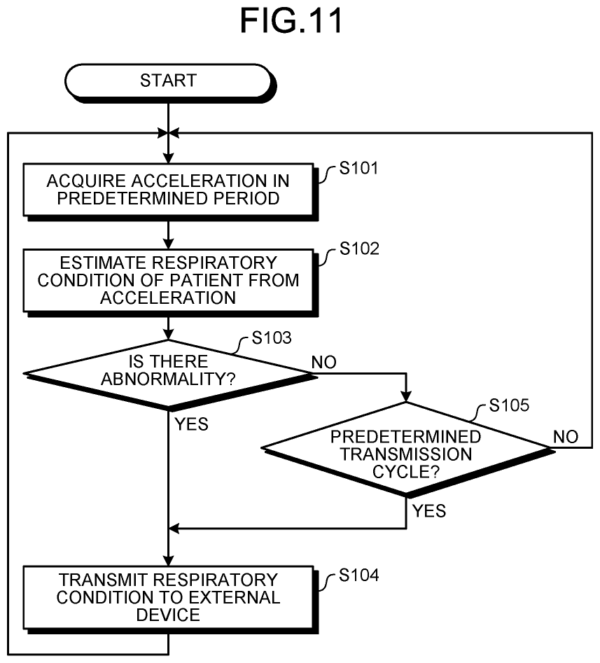

Next, a processing procedure executed by the information processing device 10 according to the embodiment will be described with reference to FIG. 11. FIG. 11 is a flowchart illustrating the processing procedure of the information processing device 10 according to the embodiment of the present disclosure.

As illustrated in FIG. 11, first, the acquisition unit 163a acquires acceleration in a predetermined period (Step S101). Then, the estimation unit 163b estimates a respiratory condition of a patient from the acceleration (Step S102).

Then, the determination unit 163c determines whether a result of the estimation is abnormal (Step S103). Here, in a case where there is abnormality (Step S103, Yes), the determination unit 163c causes the LPWA communication unit 161 to transmit the respiratory condition to the external device (Step S104). Then, the processing from Step S101 is repeated.

In addition, in a case where there is no abnormality (Step S103, No), the determination unit 163c determines whether a predetermined transmission cycle comes (Step S105). The predetermined transmission cycle is longer than a cycle in which the estimation unit 163b estimates the respiratory condition of the patient. Note that the cycle of estimating the respiratory condition of the patient corresponds to an example of a “first cycle”. Furthermore, the predetermined transmission cycle corresponds to an example of a “second cycle”.

Here, in a case where the predetermined transmission cycle comes (Step S105, Yes), the determination unit 163c causes the LPWA communication unit 161 to transmit the respiratory condition to the external device (Step S104). Then, the processing from Step S101 is repeated.

On the other hand, in a case where the predetermined transmission cycle does not come yet (Step S105, No), the determination unit 163c repeats the processing from Step S101 without causing the LPWA communication unit 161 to transmit the respiratory condition to the external device.

### 4. Modification Example

Note that there are some modification examples for the above-described embodiment.

### 4-1. First Modification Example

As the first modification example, for example, it may be determined whether a patient is in a moving state, and estimation models may be switched depending on whether the patient is in the moving state. For example, determination may be made with a first estimation model when the patient is in a non-moving state, and with a second estimation model when the patient is in the moving state.

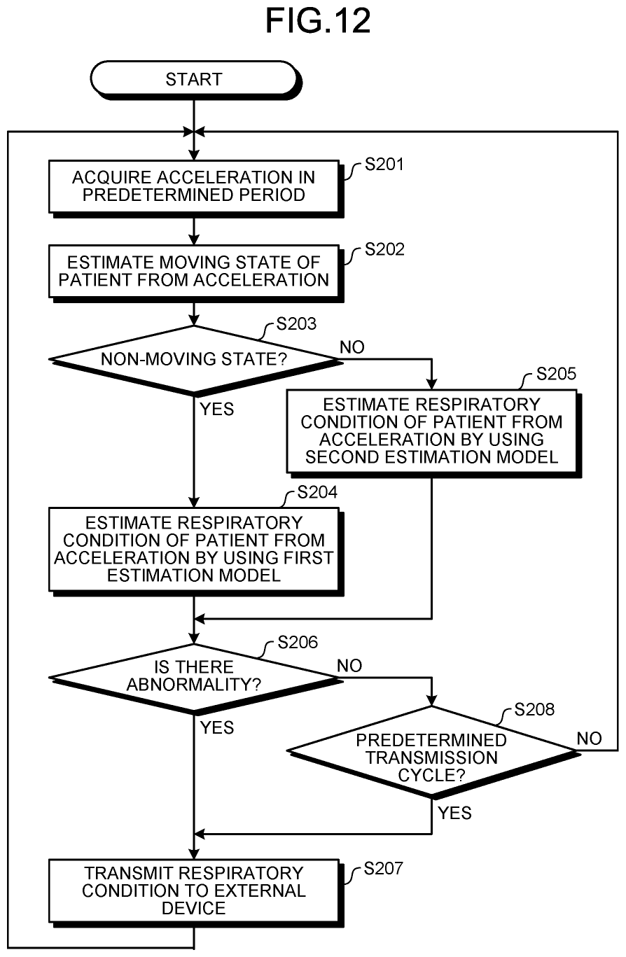

FIG. 12 is a flowchart illustrating a processing procedure of an information processing device 10 according to the first modification example. As illustrated in FIG. 12, first, an acquisition unit 163a acquires acceleration in a predetermined period (Step S201). Then, an estimation unit 163b estimates a moving state of a patient from the acceleration (Step S202).

Then, a determination unit 163c determines whether the patient is in a non-moving state (Step S203). Here, in a case where the patient is in the non-moving state (Step S203, Yes), the estimation unit 163b estimates a respiratory condition of the patient from the acceleration by using the first estimation model (Step S204).

On the other hand, in a case where the patient is in the moving state (Step S203, No), the estimation unit 163b estimates the respiratory condition of the patient from the acceleration by using the second estimation model (Step S205).

Then, the determination unit 163c determines whether a result of the estimation is abnormal (Step S206). Here, in a case where there is abnormality (Step S206, Yes), the determination unit 163c causes an LPWA communication unit 161 to transmit the respiratory condition to an external device (Step S207). Then, the processing from Step S201 is repeated.

In addition, in a case where there is no abnormality (Step S206, No), the determination unit 163c determines whether a predetermined transmission cycle comes (Step S208).

Here, in a case where the predetermined transmission cycle comes (Step S208, Yes), the determination unit 163c causes the LPWA communication unit 161 to transmit the respiratory condition to the external device (Step S207). Then, the processing from Step S201 is repeated.

On the other hand, in a case where the predetermined transmission cycle does not come yet (Step S208, No), the determination unit 163c repeats the processing from Step S201 without causing the LPWA communication unit 161 to transmit the respiratory condition to the external device.

Note that only “being in the moving state” may be transmitted and the respiratory condition may not be estimated when the patient is in the moving state. For example, when the moving state is a running state, variance is too large and it is difficult to measure the acceleration. When the moving state is a walking state, the acceleration can be measured.

### 4-2. Second Modification Example

As the second modification example, for example, estimation models may be switched on the basis of user information. In such a case, for example, an age of a patient is acquired from medical record information included in the user information illustrated in FIG. 5, and an integration server 100 selects an estimation model corresponding to the age. Then, the integration server 100 instructs an information processing device 10 to switch the estimation model used for estimation processing.

Note that such switching of the estimation model may be performed also on the basis of whether the patient is after surgery, or the like. Specifically, after the surgery, it is preferable to perform switching to an estimation model with parameters that are more sensitive (that is, weak against noise but has high sensitivity) to a respiratory change of the patient than usual since a condition of the patient is not stable.

Furthermore, instead of switching the estimation model, a period of estimating a respiratory condition of the patient (that is, “first cycle”) may be changed on the basis of the medical record information. For example, since the condition of the patient is unstable after the surgery, a respiratory condition may be estimated once every one minute for the patient after the surgery while a respiratory condition may be estimated once every five minutes for a patient who is not after surgery. Similarly, a predetermined transmission cycle (that is, “second period”) may be changed.

In addition, an estimation model or an estimation period may be switched between a patient in an intensive care unit (ICU) and a patient in a general hospital room. A type of a hospital room may be recognized on the basis of the medical record information or may be recognized from information of a base station device 50 installed in each hospital room.

In addition, as a change of the estimation model, a type of a respiratory condition to be estimated may be changed, or a type of respiration regarded as abnormal may be changed. For example, since being prone to polypnea, a patient after surgery may be considered to be normal instead of being abnormal even when being estimated to be polypnea.

### 4-3. Third Modification Example

As the third modification example, when a respiratory condition is abnormal, a degree of urgency of the abnormality may be estimated. In such a case, for example, the degree of urgency of the abnormality may be set in stages such as emergent abnormality, normal abnormality, and predictive abnormality, and information may be immediately transmitted in a case where the abnormality is the emergent abnormality. In addition, in the case of the normal abnormality or the predictive abnormality, the information may be transmitted at least before a predetermined transmission cycle comes, and the information may be transmitted at least at a timing earlier than that of the predictive abnormality in a case of the normal abnormality. Furthermore, with respect to the predictive abnormality, the transmission may be performed when the predetermined transmission cycle comes.

### 4-4. Fourth Modification Example

As the fourth modification example, in addition to estimation whether a respiratory condition is abnormal, a device disconnection of an information processing device 10 may be determined. In such a case, the information processing device 10 includes a temperature sensor in addition to an acceleration sensor 13, and can be realized, for example, by determination of a state of attachment of the information processing device 10 to a patient on the basis of the temperature sensor by a determination unit 163c. Here, in a case where it is determined that the information processing device 10 is not attached, wireless communication is not to be performed. As a result, power consumption due to unnecessary wireless communication can be controlled.

### 4-5. Fifth Modification Example

As the fifth modification example, an integration server 100 may compare a respiratory condition of a patient with user information and change a manner of an alert on a UI screen, for example. For example, in a case where the above-described predictive abnormality is detected, an alert may be output on the UI screen when the predictive abnormality continues 10 times for younger age, and an alert may be output on the UI screen when the predictive abnormality continues twice for older age. In addition, of course, a manner and type of the alert may be changed depending on the above-described emergent abnormality, normal abnormality, and predictive abnormality.

### 4-6. Sixth Modification Example

As the sixth modification example, an information processing device 10 may include a global positioning system (GPS) sensor, and switch an estimation model or change a transmission frequency of information on the basis of positioning information of the GPS sensor. For example, even in the same house, when a patient is in a bedroom and when the patient is bathing, a change in a respiratory condition varies greatly. Thus, where in the house the patient is may be determined by the GPS sensor, and the estimation model may be switched according to the position. In addition, since relaxed states are different between the inside and outside of the house and there is a high possibility of a moving state due to walking or the like, the estimation model may be switched similarly on the basis of the positioning information of the GPS sensor. Furthermore, a rate of estimation of the respiratory condition may be changed on the basis of the positioning information.

### 4-7. Other Modification Examples

Also, among the pieces of processing described in the above embodiment, a whole or part of the processing described to be automatically performed can be manually performed, or a whole or part of the processing described to be manually performed can be automatically performed by a known method. In addition, the processing procedures, specific names, and information including various kinds of data or parameters illustrated in the above document or in the drawings can be arbitrarily changed unless otherwise specified. For example, various kinds of information illustrated in each drawing are not limited to the illustrated information.

Also, each component of each of the illustrated devices is a functional concept, and does not need to be physically configured in the illustrated manner. That is, a specific form of distribution/integration of each device is not limited to what is illustrated in the drawings, and a whole or part thereof can be functionally or physically distributed/integrated in an arbitrary unit according to various loads and usage conditions. For example, the estimation unit 163b and the determination unit 163c illustrated in FIG. 6 may be integrated. Furthermore, for example, the distribution unit 103c and the output unit 103e illustrated in FIG. 7 may be integrated. Furthermore, for example, the integration server 100 may also serve as the information server 200.

In addition, the above-described embodiments can be arbitrarily combined in a region in which the processing contents do not contradict each other. Furthermore, the order of steps illustrated in the sequence diagram or the flowchart of the present embodiment can be changed as appropriate.

### 5. Hardware Configuration

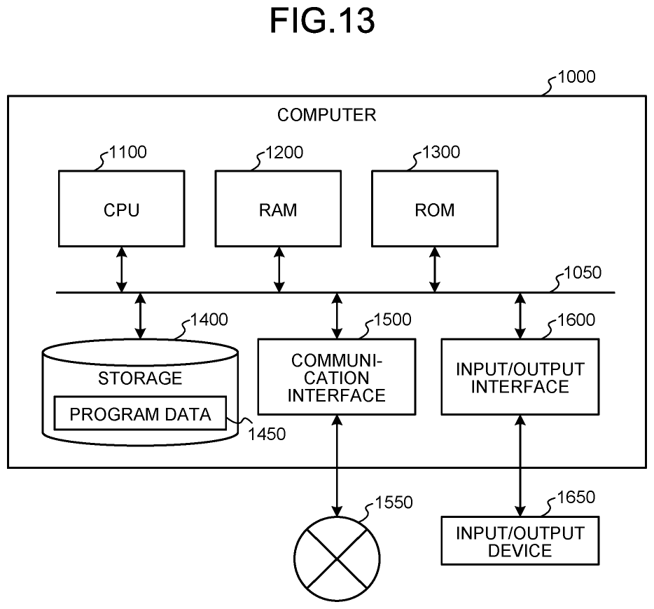

The information processing device 10, the integration server 100, the information server 200, and the terminal device 300 according to the above-described embodiment are realized by a computer 1000 having a configuration in a manner illustrated in FIG. 13, for example. The information processing device 10 will be described as an example. FIG. 13 is a hardware configuration diagram illustrating an example of the computer 1000 that realizes functions of the information processing device 10. The computer 1000 includes a CPU 1100, a RAM 1200, a ROM 1300, a storage 1400, a communication interface 1500, and an input/output interface 1600. Each unit of the computer 1000 is connected by a bus 1050.

The CPU 1100 operates on the basis of programs stored in the ROM 1300 or the storage 1400, and controls each unit. For example, the CPU 1100 expands the programs, which are stored in the ROM 1300 or the storage 1400, in the RAM 1200 and executes processing corresponding to the various programs.

The ROM 1300 stores a boot program such as a basic input output system (BIOS) executed by the CPU 1100 during activation of the computer 1000, a program that depends on hardware of the computer 1000, and the like.

The storage 1400 is a computer-readable recording medium that non-temporarily records a program executed by the CPU 1100, data used by the program, and the like. Specifically, the storage 1400 is a recording medium that records the information processing program according to the present disclosure which program is an example of program data 1450.

The communication interface 1500 is an interface with which the computer 1000 is connected to an external network 1550 (such as wireless network with the base station device 50). For example, the CPU 1100 receives data from another equipment or transmits data generated by the CPU 1100 to another equipment via the communication interface 1500.

The input/output interface 1600 is an interface to connect an input/output device 1650 and the computer 1000. For example, the CPU 1100 can receive data from an input device such as a keyboard, mouse, or the acceleration sensor 13 via the input/output interface 1600. Furthermore, the CPU 1100 can transmit data to an output device such as a display, speaker, or printer via the input/output interface 1600. Also, the input/output interface 1600 may function as a medium interface that reads a program or the like recorded on a predetermined recording medium (medium). The medium is, for example, an optical recording medium such as a digital versatile disc (DVD) or phase change rewritable disk (PD), a magneto-optical recording medium such as a magneto-optical disk (MO), a tape medium, a magnetic recording medium, a semiconductor memory, or the like.

For example, in a case where the computer 1000 functions as the information processing device 10 according to the embodiment, the CPU 1100 of the computer 1000 realizes a function of the control unit 163 by executing the information processing program loaded on the RAM 1200. Also, the storage 1400 stores the information processing program according to the present disclosure, and data in the storage unit 162. Note that the CPU 1100 reads the program data 1450 from the storage 1400 and performs execution thereof, and may acquire these programs from another device via the external network 1550 in another example.

### 6. Conclusion

As described above, according to an embodiment of the present disclosure, the information processing device 10 is an information processing device attached to a patient, and includes the acceleration sensor 13 (corresponding to an example of a “sensor”) that detects movement of the patient which movement is associated with respiratory movement, the acquisition unit 163a that acquires sensor data of the acceleration sensor 13, the estimation unit 163b that estimates a respiratory condition of the patient from the sensor data, and the determination unit 163c that determines whether to transmit information related to the respiratory condition to the integration server 100 (corresponding to an example of an “external device”) on the basis of the estimated respiratory condition. As a result, it is possible to monitor the respiratory condition of the patient while controlling the power consumption and securing the real-time property.

Although embodiments of the present disclosure have been described above, the technical scope of the present disclosure is not limited to the above-described embodiments as they are, and various modifications can be made within the spirit and scope of the present disclosure. Also, components of different embodiments and modification examples may be arbitrarily combined.

Also, an effect in each of the embodiments described in the present description is merely an example and is not a limitation, and there may be a different effect.

Note that the present technology can also have the following configurations.
(1)
An information processing device attached to a patient, the information processing device comprising:
a sensor that detects movement of the patient which movement is associated with respiratory movement;
an acquisition unit that acquires sensor data of the sensor;
an estimation unit that estimates a respiratory condition of the patient from the sensor data; and
a determination unit that determines whether to transmit information related to the respiratory condition to the an external device on a basis of the estimated respiratory condition.
(2)
The information processing device according to (1), further comprising
a wireless communication unit that changes a connection state with respect to the external device according to a result of the determination by the determination unit.
(3)
The information processing device according to (2), wherein
the wireless communication unit performs wireless communication according to an LPWA system.
(4)
The information processing device according to (2) or (3), wherein
the estimation unit
estimates the respiratory condition by using an estimation model generated by machine learning.
(5)
The information processing device according to (4), wherein
the estimation unit
estimates the respiratory condition according to a moving state of the patient by using a different estimation model.
(6)
The information processing device according to any one of (2) to (5), wherein
the estimation unit
estimate the respiratory condition in a first cycle, and
the determination unit
causes the wireless communication unit to transmit information related to the respiratory condition in a second cycle longer than the first cycle.
(7)
The information processing device according to (6), wherein
the determination unit
causes the wireless communication unit to transmit the information related to the respiratory condition before the second cycle comes in a case where the respiratory condition estimated by the estimation unit indicates abnormality.
(8)
The information processing device according to (7), wherein
the determination unit
causes the wireless communication unit to transmit the information related to the respiratory condition immediately in a case where the respiratory condition estimated by the estimation unit indicates emergent abnormality.
(9)
The information processing device according to (6), (7) or (8), wherein
the determination unit
causes the wireless communication unit to transmit, in a case where the respiratory condition estimated by the estimation unit is normal, only information indicating the normality as the information related to the respiratory condition.
(10)
The information processing device according to any one of (6) to (9), wherein
the determination unit
causes the wireless communication unit to transmit, in a case where the respiratory condition estimated by the estimation unit indicates abnormality, information including the sensor data as the information related to the respiratory condition.
(11)
The information processing device according to any one of (6) to (10), wherein
the estimation unit
can change one or both of the first cycle and the second cycle on a basis of medical record information of the patient.
(12)
The information processing device according to any one of (1) to (11), wherein
the sensor is an acceleration sensor.
(13)
The information processing device according to any one of (1) to (12), further comprising
a temperature sensor, wherein
the determination unit
determines a state of attachment to the patient on a basis of the temperature sensor, and does not transmit information related to the respiratory condition in a case where it is determined that the information processing device is not attached.
(14)
An information processing system comprising:
the information processing device according to any one of (1) to (13); and
a server device that is the external device, wherein
the server device
estimates, in a case where the respiratory condition estimated by the estimation unit indicates abnormality, the respiratory condition in more detail than the information processing device on the basis of the sensor data transmitted from the information processing device.
(15)
The information processing system according to (14), further comprising
a terminal device used by a medical worker, wherein
the server device
generates output information related to the patient on a basis of the information that is related to the respiratory condition and transmitted from the information processing device, and outputs the output information to the terminal device.
(16)
The information processing system according to (15), wherein
the output information includes an alert of a case where the respiratory condition indicates the abnormality, and
the server device
changes the alert according to a combination of user information including medical record information of the patient and the respiratory condition.
(17)
An information processing method using an information processing device that includes a sensor to detect movement of a patient which movement is associated with respiratory movement, and that is attached to the patient, the information processing method comprising:
acquiring sensor data of the sensor;
estimating a respiratory condition of the patient from the sensor data; and
determining whether to transmit information related to the respiratory condition to an external device on a basis of the estimated respiratory condition.
(18)
An information processing program causing a computer that includes a sensor to detect movement of a patient which movement is associated with respiratory movement, and that is attached to the patient to function, the information processing program causing
the computer to execute
acquiring sensor data of the sensor,
estimating a respiratory condition of the patient from the sensor data, and
determining whether to transmit information related to the respiratory condition to an external device on a basis of the estimated respiratory condition.

### REFERENCE SIGNS LIST

1 INFORMATION PROCESSING SYSTEM

10 INFORMATION PROCESSING DEVICE

13 ACCELERATION SENSOR

15 BATTERY

16 MICROCOMPUTER

161 LPWA COMMUNICATION UNIT

162 STORAGE UNIT

162a ESTIMATION MODEL

163 CONTROL UNIT

163a ACQUISITION UNIT

163b ESTIMATION UNIT

163c DETERMINATION UNIT

50 BASE STATION DEVICE

100 INTEGRATION SERVER

101 COMMUNICATION UNIT

102 STORAGE UNIT

102a ESTIMATION MODEL DB

103 CONTROL UNIT

103a ACQUISITION UNIT

103b LEARNING UNIT

103c DISTRIBUTION UNIT

103d ESTIMATION UNIT

103e OUTPUT UNIT

200 INFORMATION SERVER

201 USER INFORMATION DB

300 TERMINAL DEVICE

1. An information processing device attached to a patient, the information processing device comprising:
a sensor that detects movement of the patient which movement is associated with respiratory movement;
an acquisition unit that acquires sensor data of the sensor;
an estimation unit that estimates a respiratory condition of the patient from the sensor data; and
a determination unit that determines whether to transmit information related to the respiratory condition to the an external device on a basis of the estimated respiratory condition.

2. The information processing device according to claim 1, further comprising
a wireless communication unit that changes a connection state with respect to the external device according to a result of the determination by the determination unit.

3. The information processing device according to claim 2, wherein
the wireless communication unit performs wireless communication according to an LPWA system.

4. The information processing device according to claim 1, wherein
the estimation unit
estimates the respiratory condition by using an estimation model generated by machine learning.

5. The information processing device according to claim 4, wherein
the estimation unit
estimates the respiratory condition according to a moving state of the patient by using a different estimation model.

6. The information processing device according to claim 2, wherein
the estimation unit
estimate the respiratory condition in a first cycle, and
the determination unit
causes the wireless communication unit to transmit information related to the respiratory condition in a second cycle longer than the first cycle.

7. The information processing device according to claim 6, wherein
the determination unit
causes the wireless communication unit to transmit the information related to the respiratory condition before the second cycle comes in a case where the respiratory condition estimated by the estimation unit indicates abnormality.

8. The information processing device according to claim 7, wherein
the determination unit
causes the wireless communication unit to transmit the information related to the respiratory condition immediately in a case where the respiratory condition estimated by the estimation unit indicates emergent abnormality.

9. The information processing device according to claim 6, wherein
the determination unit
causes the wireless communication unit to transmit, in a case where the respiratory condition estimated by the estimation unit is normal, only information indicating the normality as the information related to the respiratory condition.

10. The information processing device according to claim 6, wherein
the determination unit
causes the wireless communication unit to transmit, in a case where the respiratory condition estimated by the estimation unit indicates abnormality, information including the sensor data as the information related to the respiratory condition.

11. The information processing device according to claim 6, wherein
the estimation unit
can change one or both of the first cycle and the second cycle on a basis of medical record information of the patient.

12. The information processing device according to claim 1, wherein
the sensor is an acceleration sensor.

13. The information processing device according to claim 1, further comprising
a temperature sensor, wherein
the determination unit
determines a state of attachment to the patient on a basis of the temperature sensor, and does not transmit information related to the respiratory condition in a case where it is determined that the information processing device is not attached.

14. An information processing system comprising:
the information processing device according to claim 1; and
a server device that is the external device, wherein
the server device
estimates, in a case where the respiratory condition estimated by the estimation unit indicates abnormality, the respiratory condition in more detail than the information processing device on the basis of the sensor data transmitted from the information processing device.

15. The information processing system according to claim 14, further comprising
a terminal device used by a medical worker, wherein
the server device
generates output information related to the patient on a basis of the information that is related to the respiratory condition and transmitted from the information processing device, and outputs the output information to the terminal device.

16. The information processing system according to claim 15, wherein
the output information includes an alert of a case where the respiratory condition indicates the abnormality, and
the server device
changes the alert according to a combination of user information including medical record information of the patient and the respiratory condition.

17. An information processing method using an information processing device that includes a sensor to detect movement of a patient which movement is associated with respiratory movement, and that is attached to the patient, the information processing method comprising:
acquiring sensor data of the sensor;
estimating a respiratory condition of the patient from the sensor data; and
determining whether to transmit information related to the respiratory condition to an external device on a basis of the estimated respiratory condition.

18. An information processing program causing a computer that includes a sensor to detect movement of a patient which movement is associated with respiratory movement, and that is attached to the patient to function, the information processing program causing
the computer to execute
acquiring sensor data of the sensor,
estimating a respiratory condition of the patient from the sensor data, and
determining whether to transmit information related to the respiratory condition to an external device on a basis of the estimated respiratory condition.
